(19) United States
(12) Patent Application Publication
(10) Pub. No.: US 2023/0181055 A1
(43) Pub. Date: Jun. 15, 2023

(54) Endoscope with Pressure Recorder

(71) Applicants: The Medical College of Wisconsin, Inc., Milwaukee, WI (US); University of Southern California, Los Angeles, CA (US)

(72) Inventors: Reza Shaker, Brookfield, WI (US); Anisa Shaker, Los Angeles, CA (US)

(21) Appl. No.: 18/079,714

(22) Filed: Dec. 12, 2022

Related U.S. Application Data
(60) Provisional application No. 63/289,022, filed on Dec. 13, 2021.

(51) Int. Cl. A61B 5/03; A61B 1/00; A61B 1/015; A61M 13/00; A61B 1/273

(52) U.S. Cl. CPC A61B 5/037; A61B 1/00165; A61B 1/015; A61M 13/003; A61B 1/0005; A61B 1/2736; A61M 2025/0002

(57) ABSTRACT

Disclosed is an endoscope for measuring intra-organ pressure (e.g., intragastric pressure), and more particularly an endoscope for measuring intra-organ pressure (e.g., intragastric pressure) and visualizing the response of an adjacent sphincter (e.g., lower esophageal sphincter) to changes in the intra-organ pressure.

### CROSS-REFERENCES TO RELATED APPLICATIONS

This application claims priority to U.S. Application No. 63/289,022 filed Dec. 13, 2021.

### STATEMENT REGARDING FEDERALLY SPONSORED RESEARCH

Not Applicable.

### BACKGROUND OF THE INVENTION

### 1. Field of the Invention

This invention relates to an endoscope for measuring intra-organ pressure, and more particularly to an endoscope for measuring intra-organ pressure and visualizing the response of an adjacent sphincter to changes in the intra-organ pressure.

### 2. Description of the Related Art

Gastroesophageal reflux disease (GERD) is one of the most common gastrointestinal diseases. One cause of GERD is a malfunction of the lower esophageal sphincter (LES), which controls the flow of contents between the esophagus and the stomach. The LES acts as a barrier against reflux of gastric contents into the esophagus while allowing passage of esophageal contents into the stomach. One of the malfunctions in GERD is relaxations of the LES, resulting in conditions that allow the contents of the stomach to reflux into the esophagus causing various esophageal symptoms and mucosal damage. One existing method for GERD diagnosis is endoscopy.

In endoscopic investigation, the physician introduces an endoscope, which is a thin and long tube with a display at the proximal end, through the esophagus to observe the mucosal layer of the gastrointestinal tract. The endoscopic approach mainly investigates the morphological change in the LES region due to the acidic damage done by GERD. However, current endoscopy techniques do not assess the opening and closing of the LES. As a result, endoscopic methods are unable to provide quantitative analysis about the intragastric pressure level that opens the LES.

Therefore, further improvements in endoscopic gastroesophageal reflux diagnostic technology are needed which can directly and quantitatively measure the intragastric pressure level that opens the lower esophageal sphincter.

### SUMMARY OF THE INVENTION

The present invention provides an endoscope for measuring intra-organ pressure (e.g., intragastric pressure), and more particularly to an endoscope for measuring intra-organ pressure (e.g., intragastric pressure) and visualizing the response of an adjacent sphincter (e.g., lower esophageal sphincter) to changes in the intra-organ pressure.

In one aspect of the present disclosure, there is provided an endoscope which comprises: a body defining a first lumen, the body having a distal end; a source of a fluid, the source being in fluid communication with a proximal end of the first lumen; a pressure sensor located on the distal end of the body; and a controller in electrical communication with the pressure sensor, the controller receiving electrical signals from the pressure sensor and converting the electrical signals into numerical pressure values.

In one embodiment, the endoscope further comprises a visualization device for displaying the numerical pressure values. In another embodiment, the endoscope further comprises an image carrying fiber positioned in a second lumen defined by the body; and a light transmitting guide positioned in a third lumen defined by the body, wherein the visualization device displays images received from the image carrying fiber. In another embodiment, the endoscope further comprises an apparatus for controlling deflection of the distal end of the endoscope. In another embodiment, the controller executes a program stored in the controller to record the numerical pressure values. In another embodiment, the endoscope further comprises a switch in electrical communication with the controller, wherein the controller executes the program stored in the controller to record a numerical pressure value when the switch is activated.

In another aspect of the present disclosure, there is provided an endoscope which comprises: a body defining a first lumen, the body having a distal end; an insufflation catheter positioned in the first lumen and movable within the first lumen, the insufflation catheter being dimensioned to advance beyond the distal end of the body; a pressure sensing catheter positioned in an additional lumen defined by the body, the pressure sensing catheter movable within the additional lumen, the pressure sensing catheter being dimensioned to advance beyond the distal end of the body; and a controller in electrical communication with the pressure sensing catheter, the controller receiving electrical signals from the pressure sensing catheter and converting the electrical signals into numerical pressure values.

In one embodiment, the endoscope further comprises a visualization device for displaying the numerical pressure values. In another embodiment, the endoscope further comprises an image carrying fiber positioned in a second additional lumen defined by the body; and a light transmitting fiber positioned in a third additional lumen defined by the body, wherein the visualization device displays images received from the image carrying fiber. In another embodiment, the controller executes a program stored in the controller to record the numerical pressure values.

In another embodiment, the endoscope further comprises a switch in electrical communication with the controller, wherein the controller executes the program stored in the controller to record a numerical pressure value when the switch is activated.

In another aspect of the present disclosure, there is provided a method for measuring an intra-organ pressure of an organ (e.g., stomach) of a subject using an endoscope of the present disclosure. The method can comprise: (a) positioning a distal end of an endoscope in the organ, the endoscope including (i) a pressure sensor located on the distal end of a body of the endoscope, and (ii) a controller in electrical communication with the pressure sensor, the controller being configured to receive an electrical signal from the pressure sensor and convert the electrical signal into a numerical pressure value of the intra-organ pressure of the organ.

In another aspect of the present disclosure, there is provided a method for monitoring the opening and closing of a sphincter (e.g., lower esophageal sphincter) located between a first volume of a first body part (e.g., stomach) on a first side of the sphincter and a second volume of a second body part (e.g., esophagus) on an opposite second side of the sphincter of a subject using one example embodiment of an endoscope of the present disclosure. The method can comprise: (a) positioning a distal end of an endoscope in the first volume of the first body part on the first side of the sphincter, the endoscope including (i) a body defining a first lumen, the body having the distal end, (ii) a source of a fluid, the source being in fluid communication with a proximal end of the first lumen, (iii) a pressure sensor located on the distal end of the body, and (iv) a controller in electrical communication with the pressure sensor, the controller being configured to receive electrical signals from the pressure sensor and convert the electrical signals into numerical pressure values; (b) introducing the fluid into the first volume of the first body part on the first side of the sphincter at least until a time at which the sphincter opens; (c) visualizing the sphincter with the endoscope to determine the time at which the sphincter opens; and (d) recording a numerical pressure value from the controller at the time at which the sphincter opens. In one embodiment of the method, the first body part is a stomach, the sphincter is a lower esophageal sphincter, and the second body part is an esophagus. In one embodiment of the method, the endoscope includes a visualization device for displaying the numerical pressure values. In one embodiment of the method, the controller includes a switch, and the controller executes the program stored in the controller to record a numerical pressure value when the switch is activated.

In another aspect of the present disclosure, there is provided a method for monitoring the opening and closing of a sphincter (e.g., lower esophageal sphincter) located between a first volume of a first body part (e.g., stomach) on a first side of the sphincter and a second volume of a second body part (e.g., esophagus) on an opposite second side of the sphincter of a subject using another example embodiment of an endoscope of the present disclosure. The method can comprise: (a) positioning a distal end of an endoscope in the second volume of the second body part on the second side of the sphincter, the endoscope including (i) a body defining a first lumen, the body having the distal end, (ii) an insufflation catheter positioned in the first lumen and movable within the first lumen, the insufflation catheter being dimensioned to advance beyond the distal end of the body, (iii) a pressure sensing catheter positioned in an additional lumen defined by the body, the pressure sensing catheter movable within the additional lumen, the pressure sensing catheter being dimensioned to advance beyond the distal end of the body, and (iv) a controller in electrical communication with the pressure sensing catheter, the controller being configured to receive electrical signals from the pressure sensing catheter and convert the electrical signals into numerical pressure values; (b) positioning a distal end of the pressure sensing catheter in the first volume of the first body part on the first side of the sphincter; (c) positioning a distal end of the insufflation catheter in the first volume of the first body part on the first side of the sphincter; (d) introducing fluid from the insufflation catheter into the first volume of the first body part on the first side of the sphincter at least until a time at which the sphincter opens; and (e) visualizing the sphincter with the endoscope to determine the time at which the sphincter opens; and (f) recording a numerical pressure value from the controller at the time at which the sphincter opens. In one embodiment of the method, the first body part is a stomach, the sphincter is a lower esophageal sphincter, and the second body part is an esophagus. The endoscope can include a visualization device for displaying the numerical pressure values.

These and other features, aspects, and advantages of the present invention will become better understood upon consideration of the following detailed description, drawings and appended claims.

### BRIEF DESCRIPTION OF THE DRAWINGS

FIG. 1 is a partial cross-sectional view of a distal portion of one example embodiment of an endoscope according to the present disclosure;

FIG. 2 is a cross-sectional view of the endoscope of FIG. 1 positioned to provide quantitative analysis about the intragastric pressure level that opens the lower esophageal sphincter (LES);

FIG. 3 is a partial cross-sectional view of a distal portion of another example embodiment of an endoscope according to the present disclosure; and

FIG. 4 is a cross-sectional view of the endoscope of FIG. 3 positioned to provide quantitative analysis about the intragastric pressure level that opens the lower esophageal sphincter (LES).

Like reference numerals will be used to refer to like parts from Figure to Figure in the following description of the drawings.

### DETAILED DESCRIPTION OF THE INVENTION

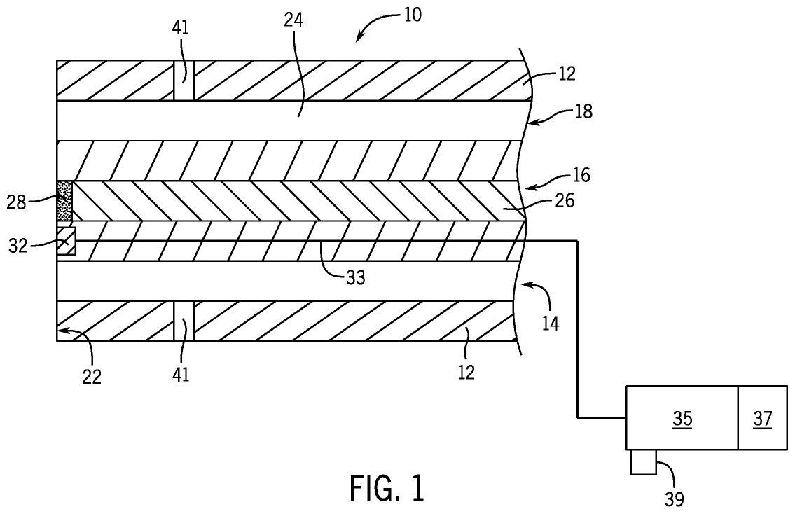

Referring to FIG. 1, there is shown a partial cross-sectional view of a distal portion of one example embodiment of an endoscope 10 according to the present invention. The endoscope 10 has a body 12 defining a first lumen 14 a second lumen 16, and a third lumen 18. The body 12 may comprise a flexible polymeric material, such as a silicone, a polyurethane, or a polyester. The endoscope 10 has a distal end 22. The first lumen 14 provides a passageway for insufflating fluid from the distal end 22 wherein the fluid (e.g., air or water) is provided from a fluid source in fluid communication with a proximal end of the first lumen 14.

A light transmitting guide 24 is positioned in the third lumen 18 for providing light to the surface area being viewed. An image carrying fiber 26 is positioned in the second lumen 16. A lens 28 is positioned at a distal end of an image carrying fiber 26, and the lens 28 is optically connected to the image carrying fiber 26 for receiving light that has been reflected from the surface area being viewed.

A pressure sensor 32 is positioned at a surface of the distal end 22 of the endoscope 10. A wire 33 places the pressure sensor 32 in electrical communication with a controller 35. The pressure sensor 32 can be a strain gauge based pressure sensor that converts strain from pressure to electrical signals.

The distal end 22 of the endoscope 10 can be maneuvered using an apparatus 41 that controls the deflection of the distal end 22 of the endoscope 10. One example apparatus to control the deflection of the distal end 22 of the endoscope 10 can be found in U.S. Pat. No. 4,503,842 which is incorporated herein by reference. Another example apparatus to control the deflection of the distal end 22 of the endoscope 10 comprises a first set of a plurality of rings connected to each other by pivot pins which allow the plurality of rings to curl the distal end 22 in the up-and-down direction. A similar second set of pivoting rings allows the distal end 22 to curl in the right-and-left direction. Together, they enable the distal end 22 to curl in any direction. The direction of the curl of the distal end 22 can be controlled by four angulation wires attached to the distal end 22 at 90 degree spacing. Pulling on one or more of the wires causes the distal end 22 to curl in any direction.

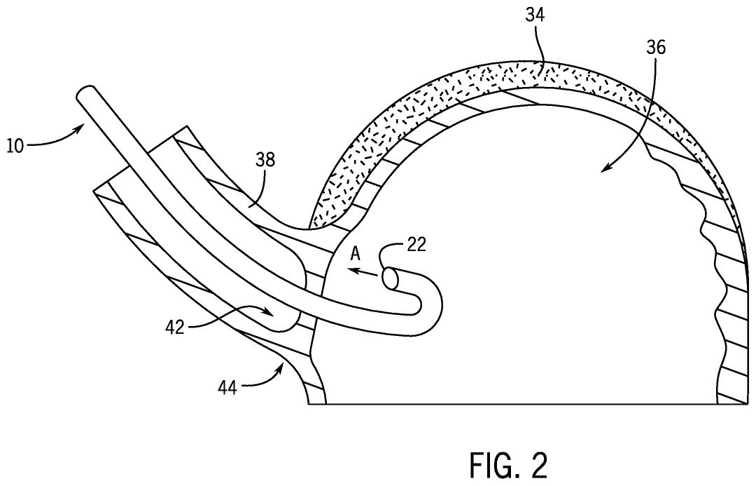

Having described components of the endoscope 10, an example method of operation of the endoscope 10 can be explained with reference to FIG. 2. The endoscope 10 is advanced through an interior volume 42 of an esophagus 38 and then through the lower esophageal sphincter (LES) 44 such that the distal end 22 of the endoscope 10 is positioned within an interior volume 36 of the stomach 34. The distal end 22 of the endoscope 10 is maneuvered using apparatus 41 such that the distal end 22 faces the LES 44 in a viewing direction A.

Fluid for insufflation is then introduced from the first lumen 14 into the interior volume 36 of the stomach 34, thereby raising intragastric pressure. Electrical signals are generated by the pressure sensor 32 in response to the pressure exerted on the pressure sensor 32. The electrical signals generated by the pressure sensor 32 are transmitted to the controller via the wire 33. A processor within the controller executes a program to convert the electrical signals to numerical pressure values that are displayed on a display 37 of the controller 36. The program can also record the numerical pressure values as a function of time in a data storage device of the controller 36. The physician views images of the LES 44 from the image carrying fiber 26 on the display 37 and also watches the display 37 for the numerical pressure value at the time when the LES 44 opens. Alternatively, the endoscope 10 may include a switch 39 in electrical communication with the controller, and the physician presses the switch 39 at the time that the LES 44 opens as shown on the display 37. The program in the controller can record in a data storage device of the controller 36 the numerical pressure values before the time when the LES 44 opens, at the time when the LES 44 opens as indicated by the pressing of the switch 39, and after the time when the LES 44 opens.

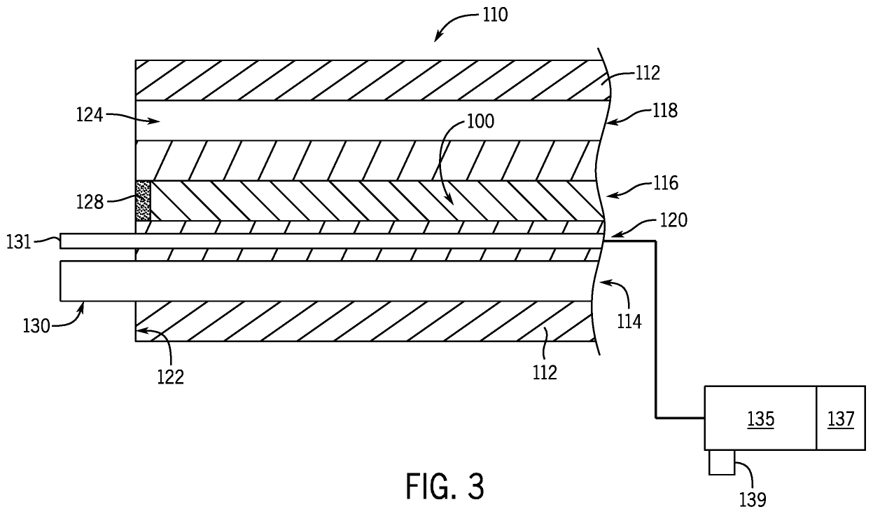

Referring now to FIG. 3, there is shown a partial cross-sectional view of a distal portion of another example embodiment of an endoscope 110 according to the present invention. The endoscope 110 has a body 112 defining a first lumen 114 a second lumen 116, a third lumen 118, and a fourth lumen 120. The body may comprise a flexible polymeric material. The endoscope 110 has a distal end 122. The first lumen 114 provides a passageway for an insufflation catheter 130 that can be advanced beyond the distal end 122 of the endoscope 110 such that insufflation fluid (e.g., air or water) can be provided from a fluid source in fluid communication with a proximal end of the insufflation catheter 130. The insufflation catheter 130 can be structured similar to the endoscope 10 except that the second lumen 16, the third lumen 18, the light transmitting guide 24, the image carrying fiber 26, the lens 28, the pressure sensor 32, and the wire 33 are omitted.

A light transmitting fiber 124 is positioned in the third lumen 118 for providing light to the surface area being viewed. An image carrying fiber 100 is positioned in the second lumen 116. A lens 128 is positioned at a distal end of an image carrying fiber 100, and the lens 128 is optically connected to the image carrying fiber 100 for receiving light that has been reflected from the surface area being viewed.

A pressure sensing catheter 131 is positioned in the fourth lumen 120, and can be advanced beyond the distal end 122 of the endoscope 110. The pressure sensing catheter 131 is in electrical communication with a controller 135. The pressure sensor of pressure sensing catheter 131 can be positioned at a surface of the distal end of the pressure sensing catheter 131. The pressure sensor can be a strain gauge based pressure sensor that converts strain from pressure to electrical signals. The pressure sensing catheter 131 can be structured similar to the endoscope 10 except that the first lumen 14, the second lumen 16, the third lumen 18, the light transmitting guide 24, the image carrying fiber 26, and the lens 28 are omitted.

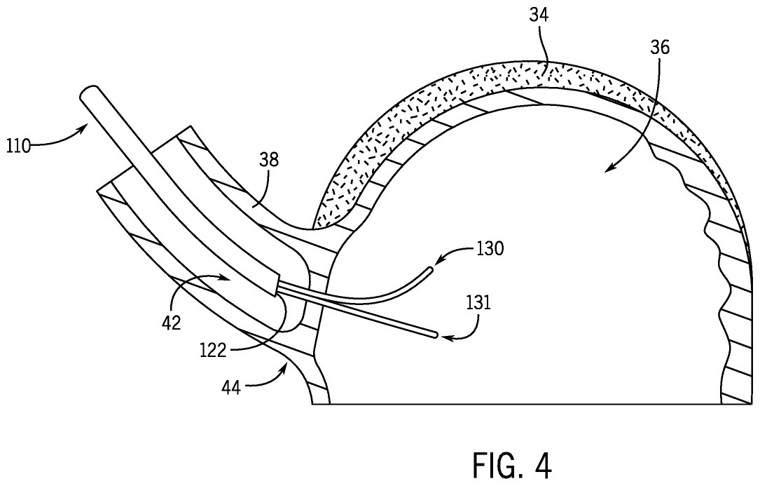

Having described components of the endoscope 110, an example method of operation of the endoscope 110 can be explained with reference to FIG. 4. The endoscope 110 is advanced through the interior volume 42 of the esophagus 38 such that the distal end 122 of the endoscope 110 is positioned within the interior volume 42 of the esophagus 38 proximal to the LES 44. The distal end of the insufflation catheter 130 is advanced beyond the distal end 122 of the endoscope 110 and through the LES 44 such that the distal end of the insufflation catheter 130 is within the interior volume 36 of the stomach 34. The pressure sensing catheter 131 is advanced beyond the distal end 122 of the endoscope 110 and through the LES 44 such that the distal end of the pressure sensing catheter 131 is within the interior volume 36 of the stomach 34.

Fluid for insufflation is then introduced from insufflation catheter 130 into the interior volume 36 of the stomach 34, thereby raising intragastric pressure. Electrical signals are generated by the pressure sensing catheter 131 in response to the pressure exerted on the pressure sensing catheter 131. The electrical signals from the pressure sensing catheter 131 are transmitted to the controller from the pressure sensing catheter 131. A processor within the controller executes a program to convert the electrical signals to numerical pressure values that are displayed on a display 137 of the controller 135. The program can also record in a data storage device of the controller 136 the numerical pressure values as a function of time. The physician views images of the LES 44 from the image carrying fiber 100 on the display 137 and also watches the display 137 for the numerical pressure value at the time when the LES 44 opens. Alternatively, the endoscope 110 may include a switch 139 in electrical communication with the controller, and the physician presses the switch 139 at the time that the LES 44 opens as shown on the display. The program in the controller can record in a data storage device of the controller 136 the numerical pressure values before the time when the LES 44 opens, at the time when the LES 44 opens as indicated by the pressing of the switch 139, and after the time when the LES 44 opens.

In other versions of the endoscopes 10 and 110, one or more additional lumens may be provided in the body of the endoscope. For example, additional lumen(s) can be provided for fluid injection used for distending the organ (e.g., stomach) and washing the debris respectively. An additional lumen can also be provided for receiving a medical instrument. Non-limiting examples of the medical instrument include a biopsy forceps, an electrocauterization device, an ablation device, and a suturing or stapling device.

While FIGS. 2 and 4 depict the measurement of intragastric pressure and visualization of the opening of the lower esophageal sphincter, the endoscopes 10 and 110 can be used to measure any intra-organ pressure and the response of an adjacent sphincter to changes in the intra-organ pressure.

Thus, the present invention provides an endoscope for measuring intra-organ pressure (e.g., intragastric pressure), and more particularly to an endoscope for measuring intra-organ pressure (e.g., intragastric pressure) and visualizing the response of an adjacent sphincter (e.g., lower esophageal sphincter) to changes in the intra-organ pressure.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment,” or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. An endoscope comprising:
a body defining a first lumen, the body having a distal end;
a source of a fluid, the source being in fluid communication with a proximal end of the first lumen;
a pressure sensor located on the distal end of the body; and
a controller in electrical communication with the pressure sensor, the controller receiving electrical signals from the pressure sensor and converting the electrical signals into numerical pressure values.

2. The endoscope of claim 1 further comprising:
a visualization device for displaying the numerical pressure values.

3. The endoscope of claim 2 further comprising:
an image carrying fiber positioned in a second lumen defined by the body; and
a light transmitting guide positioned in a third lumen defined by the body,
wherein the visualization device displays images received from the image carrying fiber.

4. The endoscope of claim 3 further comprising:
an apparatus for controlling deflection of the distal end of the endoscope.

5. The endoscope of claim 1 wherein:
the controller executes a program stored in the controller to record the numerical pressure values.

6. The endoscope of claim 5 further comprising:
a switch in electrical communication with the controller,
wherein the controller executes the program stored in the controller to record a numerical pressure value when the switch is activated.

7. An endoscope comprising:
a body defining a first lumen, the body having a distal end;
an insufflation catheter positioned in the first lumen and movable within the first lumen, the insufflation catheter being dimensioned to advance beyond the distal end of the body;
a pressure sensing catheter positioned in an additional lumen defined by the body, the pressure sensing catheter movable within the additional lumen, the pressure sensing catheter being dimensioned to advance beyond the distal end of the body; and
a controller in electrical communication with the pressure sensing catheter, the controller receiving electrical signals from the pressure sensing catheter and converting the electrical signals into numerical pressure values.

8. The endoscope of claim 7 further comprising:
a visualization device for displaying the numerical pressure values.

9. The endoscope of claim 8 further comprising:
an image carrying fiber positioned in a second additional lumen defined by the body; and
a light transmitting fiber positioned in a third additional lumen defined by the body,
wherein the visualization device displays images received from the image carrying fiber.

10. The endoscope of claim 7 wherein:
the controller executes a program stored in the controller to record the numerical pressure values.

11. The endoscope of claim 10 further comprising:
a switch in electrical communication with the controller,
wherein the controller executes the program stored in the controller to record a numerical pressure value when the switch is activated.

12. A method for measuring an intra-organ pressure of an organ of a subject, the method comprising:
(a) positioning a distal end of an endoscope in the organ, the endoscope including (i) a pressure sensor located on the distal end of a body of the endoscope, and (ii) a controller in electrical communication with the pressure sensor, the controller being configured to receive an electrical signal from the pressure sensor and convert the electrical signal into a numerical pressure value of the intra-organ pressure of the organ.

13. The method of claim 12 wherein:
the organ is a stomach.

14. A method for monitoring the opening and closing of a sphincter located between a first volume of a first body part on a first side of the sphincter and a second volume of a second body part on an opposite second side of the sphincter of a subject, the method comprising:
(a) positioning a distal end of an endoscope in the first volume of the first body part on the first side of the sphincter, the endoscope including (i) a body defining a first lumen, the body having the distal end, (ii) a source of a fluid, the source being in fluid communication with a proximal end of the first lumen, (iii) a pressure sensor located on the distal end of the body, and (iv) a controller in electrical communication with the pressure sensor, the controller being configured to receive electrical signals from the pressure sensor and convert the electrical signals into numerical pressure values;
(b) introducing the fluid into the first volume of the first body part on the first side of the sphincter at least until a time at which the sphincter opens;
(c) visualizing the sphincter with the endoscope to determine the time at which the sphincter opens; and
(d) recording a numerical pressure value from the controller at the time at which the sphincter opens.

15. The method of claim 14 wherein:
the first body part is a stomach,
the sphincter is a lower esophageal sphincter, and
the second body part is an esophagus.

16. The method of claim 14 wherein:
the endoscope includes a visualization device for displaying the numerical pressure values.

17. The method of claim 14 wherein:
the controller includes a switch, and
the controller executes the program stored in the controller to record a numerical pressure value when the switch is activated.

18. A method for monitoring the opening and closing of a sphincter located between a first volume of a first body part on a first side of the sphincter and a second volume of a second body part on an opposite second side of the sphincter of a subject, the method comprising:
(a) positioning a distal end of an endoscope in the second volume of the second body part on the second side of the sphincter, the endoscope including (i) a body defining a first lumen, the body having the distal end, (ii) an insufflation catheter positioned in the first lumen and movable within the first lumen, the insufflation catheter being dimensioned to advance beyond the distal end of the body, (iii) a pressure sensing catheter positioned in an additional lumen defined by the body, the pressure sensing catheter movable within the additional lumen, the pressure sensing catheter being dimensioned to advance beyond the distal end of the body, and (iv) a controller in electrical communication with the pressure sensing catheter, the controller being configured to receive electrical signals from the pressure sensing catheter and convert the electrical signals into numerical pressure values;
(b) positioning a distal end of the pressure sensing catheter in the first volume of the first body part on the first side of the sphincter;
(c) positioning a distal end of the insufflation catheter in the first volume of the first body part on the first side of the sphincter;
(d) introducing fluid from the insufflation catheter into the first volume of the first body part on the first side of the sphincter at least until a time at which the sphincter opens; and
(e) visualizing the sphincter with the endoscope to determine the time at which the sphincter opens; and
(f) recording a numerical pressure value from the controller at the time at which the sphincter opens.

19. The method of claim 18 wherein:
the first body part is a stomach,
the sphincter is a lower esophageal sphincter, and
the second body part is an esophagus.

20. The method of claim 18 wherein:
the endoscope includes a visualization device for displaying the numerical pressure values.
